(19) United States
(12) Patent Application Publication
(10) Pub. No.: US 2021/0076990 A1
(43) Pub. Date: Mar. 18, 2021

(54) MICROSENSOR FOR A COCHLEAR IMPLANT

(71) Applicants: Somayeh Alimohammadi, Tabriz (IR); Mohammad Imani, Tehran (IR); Mohammadali Kiani, Karaj (IR); Pezhman Sasanpour, Tehran (IR)

(72) Inventors: Somayeh Alimohammadi, Tabriz (IR); Mohammad Imani, Tehran (IR); Mohammadali Kiani, Karaj (IR); Pezhman Sasanpour, Tehran (IR)

(21) Appl. No.: 17/106,113

(22) Filed: Nov. 29, 2020

Related U.S. Application Data
(60) Provisional application No. 62/942,144, filed on Dec. 1, 2019.

(51) Int. Cl. A61B 5/145; A61B 5/1473; A61B 5/00

(52) U.S. Cl. CPC A61B 5/14507; A61B 5/1473; A61B 2560/0228; A61B 5/6817; A61B 5/14546

(57) ABSTRACT

An electrochemical system integrated with a cochlear implant to measure a corticosteroid concentration in a cochlear fluid may include a microsensor attached to an electrode array of the cochlear implant. An exemplary microsensor may be configured to be put in contact with a cochlear fluid. An exemplary microsensor may include a working electrode including a carbon microfiber with a diameter of 5 micron to 10 micron, a reference electrode including an Ag/AgCl wire with a diameter of 10 micron to 100 micron, and a counter electrode including a platinum wire with a diameter of 10 micron to 100 micron. An exemplary electrochemical system may further include an electrochemical stimulator/analyzer to measure electrochemical responses from the microsensor, and an array of electrically conductive connectors that may connect an exemplary microsensor to an exemplary electrochemical stimulator/analyzer.

### CROSS-REFERENCE TO RELATED APPLICATION

This application claims the benefit of priority from pending U.S. Provisional Patent Application Ser. No. 62/942,144, filed on Dec. 1, 2019, and entitled “INSERTABLE MICRO-SENSOR IN THE COCHLEAR IMPLANT,” which is incorporated herein by reference in its entirety.

### TECHNICAL FIELD

The present disclosure relates to cochlear implants and particularly relates to systems and methods for improving the performance of cochlear implants. More particularly, the present disclosure relates to microsensors insertable into cochlear implants for measuring concentrations of corticosteroids.

### BACKGROUND

A cochlear implant is a small electronic medical device that restores hearing in people who are profoundly deaf or hard of hearing. Cochlear implants, generally, perform the function of damaged parts of the inner ear (cochlea) to provide sound signals to the brain. Sound waves are converted into separated electric signals at different frequency bands by a cochlear implant. The electric signals produced by a cochlear implant may stimulate the auditory nerve and the brain may interpret them as sound. A cochlear implant may include a microphone for picking up environmental sounds, a stimulator that may convert sound waves received from the microphone into electric impulses, and an array of auditory electrodes that may collect the impulses produced by the stimulator and utilize them to stimulate the auditory nerve. A cochlear implant, as opposed to a hearing aid, does not amplify sounds so that they may be detected by damaged ears, instead a cochlear implant bypasses damaged portions of the ear and allows for a direct stimulation of the auditory nerves.

One of the problems with implanting medical devices, such as cochlear implants is the damage that may be incurred at implantation location, which may cause apoptosis or necrosis of the nerve tissue (capillary and spiral ganglion cells) and may limit the implant function. In order to reduce a tissue damage, the implant structure must be soft and flexible. Another major problem after implanting a cochlear implant may be an increase of the impedance of the array of auditory electrodes over time. This increase in the impedance may be mainly due to the enclosure of the array of electrodes by a fibrous inflammatory tissue, which may reduce the efficiency of the array of electrodes and contacts in the process of electrical stimulation.

As mentioned in the previous paragraph, an inflammation in tissue may surround the array of auditory electrodes and may interfere with their proper function. One way to address this problem is to administer drugs, such as corticosteroids, to reduce inflammation, and in turn to improve electrode impedance after surgery. Implant impregnation by drug during surgery is a common old method of administration as there is no direct way to transfer the medicine to the inner ear after cochlear implantation. It should be noted that the middle ear is not easily accessible. Actually, the inner ear is a closed system where the drug cannot be directly administered into.

In one approach, cochlear implants may be provided with a channel at a center of the array of electrodes passing through pores of the array of electrodes. An anti-inflammatory drug may be pumped through the aforementioned channel from a drug container to the inner ear. For example, a titanium mini pump may be utilized for pumping the anti-inflammatory drug into the inner ear. In another approach, a cochlear implant may be coated with a silicone coating that may include an anti-inflammatory drug. This way, an anti-inflammatory drug such as dexamethasone may be slowly released from the silicone coating into the inner ear. In this approach, the anti-inflammatory drug may be gradually released from the silicone coating within one to three months.

Generally, in drug delivery, the blood concentration and pharmacokinetics of the delivered drugs are important factors in determining the efficacy of the drug delivery. Accordingly, determination of the amount of an anti-inflammatory drug in the perilymph after implanting a cochlear implant is crucial and requires sensitive analysis methods. Such a requirement is independent of the method of administration and the drug may be administered to the inner ear through an electrode tip, through a single channel drug delivery device, or the drug may be released from a silicon coated cochlear implant.

Microanalysis is the only available method for sampling the cochlear fluid and determining the drug concentration after the surgery. Microanalysis may allow for measuring the drug concentration in the perilymph without changing the fluid volume or transmission of microbial contamination during sampling. However, microanalysis must be performed outside the body, and therefore, it is time consuming. Furthermore, performing microanalysis requires a specialist and only limited centers around the world have the required equipment and staff to perform microanalysis. There is, therefore, a need for fabrication of a micro-sensor that may be inserted into the structure of a cochlear implant for measuring the drug concentration quickly and without the need for a specialist or special equipment.

### SUMMARY

This summary is intended to provide an overview of the subject matter of the present disclosure and is not intended to identify essential elements or key elements of the subject matter, nor is it intended to be used to determine the scope of the claimed implementations. The proper scope of the present disclosure may be ascertained from the claims set forth below in view of the detailed description and the drawings.

According to one or more exemplary embodiments, the present disclosure is directed to an electrochemical system integrated with a cochlear implant to measure a corticosteroid concentration in a cochlear fluid. An exemplary electrochemical system may include a microsensor attached to an electrode array of the cochlear implant. An exemplary microsensor may be configured to be put in contact with a cochlear fluid. An exemplary microsensor may include a working electrode including a carbon microfiber with a diameter of 5 micron to 10 micron, a reference electrode including an Ag/AgCl wire with a diameter of 10 micron to 100 micron, and a counter electrode including a platinum wire with a diameter of 10 micron to 100 micron. An exemplary electrochemical system may further include an electrochemical stimulator/analyzer to measure electrochemical responses from the microsensor, and an array of electrically conductive connectors that may connect an exemplary microsensor to an exemplary electrochemical stimulator/analyzer.

In an exemplary embodiment, an exemplary electrochemical system may further include a processing unit that may be coupled with an exemplary electrochemical stimulator/analyzer. An exemplary processing unit may include at least one processor, and at least one memory that may be coupled to the at least one processor. At least one exemplary memory may store executable instructions to urge an exemplary processor to receive a measured electrochemical response from an exemplary electrochemical stimulator/analyzer, receive a calibration relationship between a peak current of the measured electrochemical response and a corticosteroid concentration in a cochlear fluid, and calculate a corticosteroid concentration in a cochlear fluid based at least in part on a measured electrochemical response utilizing a received calibration relationship between a peak current of the measured electrochemical response and a corticosteroid concentration in a cochlear fluid.

### BRIEF DESCRIPTION OF THE DRAWINGS

The novel features which are believed to be characteristic of the present disclosure, as to its structure, organization, use and method of operation, together with further objectives and advantages thereof, will be better understood from the following drawings in which a presently preferred embodiment of the present disclosure will now be illustrated by way of example. It is expressly understood, however, that the drawings are for illustration and description only and are not intended as a definition of the limits of the present disclosure. Embodiments of the present disclosure will now be described by way of example in association with the accompanying drawings in which:

FIG. 1 illustrates a cochlear implant system, consistent with one or more exemplary embodiments of the present disclosure;

FIG. 2A illustrates a schematic top view of a human cochlea and a lead of a cochlear implant system inserted into a human cochlea, consistent with one or more exemplary embodiments of the present disclosure;

FIG. 2B illustrates a schematic structure of a lead, consistent with one or more exemplary embodiments of the present disclosure;

FIG. 3A illustrates a box diagram of an electrochemical system, consistent with one or more exemplary embodiments of the present disclosure;

FIG. 3B illustrates a high-level functional block diagram of a processing unit, consistent with one or more exemplary embodiments of the present disclosure;

FIGS. 4A-4D illustrate an exemplary electrochemical sensor at different stages of manufacturing process, consistent with one or more exemplary embodiments of the present disclosure;

FIG. 5A illustrates a scanning electron microscope (SEM) image of a single carbon fiber before surface treatment, consistent with one or more exemplary embodiments of the present disclosure;

FIG. 5B illustrates an SEM of a single carbon fiber after surface treatment, consistent with one or more exemplary embodiments of the present disclosure;

FIG. 6 illustrates differential pulse voltammetry (DPV) voltammograms obtained by an electrochemical system for measuring 50 μM of dexamethasone in an artificial perilymph solution at different pulse amplitudes, consistent with one or more exemplary embodiments of the present disclosure;

FIG. 7 illustrates DPV voltammograms 70 obtained by an electrochemical system for measuring 50 μM of dexamethasone in an artificial perilymph solution at different pulse times, consistent with one or more exemplary embodiments of the present disclosure;

FIG. 8 illustrates DPV voltammograms obtained by an electrochemical system for measuring 50 μM of dexamethasone in an artificial perilymph solution at different potential steps, consistent with one or more exemplary embodiments of the present disclosure;

FIG. 9 illustrates DPV voltammograms obtained from an electrochemical sensor for various artificial perilymph solutions containing different amounts of dexamethasone, consistent with one or more exemplary embodiments of the present disclosure;

FIG. 10 illustrates calibration plots of peak currents versus various concentrations of dexamethasone in artificial perilymph solutions, consistent with one or more exemplary embodiments of the present disclosure.

### DETAILED DESCRIPTION

The novel features which are believed to be characteristic of the present disclosure, as to its structure, organization, use and method of operation, together with further objectives and advantages thereof, will be better understood from the following discussion.

The present disclosure is directed to exemplary embodiments of an exemplary electrochemical system that may be associated with a cochlear implant for measuring the concentration of an anti-inflammatory drug administered into an inner ear of an implantee during or after the implantation of the cochlear implant. An exemplary cochlear implant may include a lead with a plurality of electrodes arranged thereon that may be implanted within a human cochlea. The presence of an exemplary lead of an exemplary cochlear implant within a human cochlea may cause inflammation in the tissue of the human cochlea. Such inflammation, as was discussed earlier may surround exemplary electrodes of an exemplary lead and may affect their performance. It is therefore necessary to reduce the inflammation within the cochlea. To this end, an anti-inflammatory drug must be administered into the cochlea and the concentration of this anti-inflammatory drug must be monitored to ensure an efficient drug delivery. An exemplary electrochemical system may be associated with the cochlear implant to monitor the concentration of an exemplary anti-inflammatory drug easily and effortlessly within cochlear fluid.

An exemplary electrochemical system may include an electrochemical microsensor that may be attached to an exemplary lead of an exemplary cochlear implant. An exemplary electrochemical microsensor may be implanted along with an exemplary lead within a human cochlea. An exemplary electrochemical microsensor may be coupled to an electrochemical stimulator/analyzer outside an implantee's body and may be configured to measure the concentration of an anti-inflammatory drug within cochlear fluid.

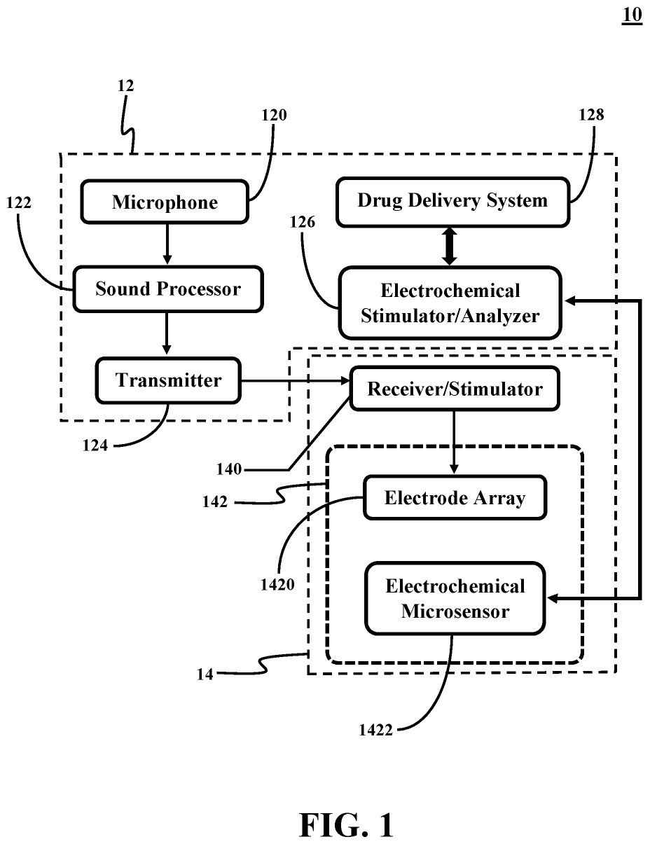

FIG. 1 illustrates a cochlear implant system 10, consistent with one or more exemplary embodiments of the present disclosure. In an exemplary embodiment, system 10 may include an external section 12 that may be configured to be located external to an implantee. For example, external section 12 may be configured to be located on the skin behind an ear of an implantee. In an exemplary embodiment, components of external section 12 may include, but may not be limited to, a microphone 120, a sound processor 122, a transmitter 124, and an electrochemical stimulator/analyzer 126. In an exemplary embodiment, system 10 may further include an implanted section 14 that may be configured to be implanted inside an implantee. As used herein, inside an implantee may refer to an inner ear of an implantee and under the skin behind an ear of the implantee. In an exemplary embodiment, components of implanted section 14 may include, but not limited to, a receiver/stimulator 140 and a lead 142. In an exemplary embodiment, lead 142 may include an electrode array 1420 and an electrochemical microsensor 1422. In an exemplary embodiment, system 10 may further include additional or alternative components that may serve a particular embodiment, as will be described later. For example, system 10 may further include a drug delivery system 128 in external section 12. In an exemplary embodiment, some components within system 10 may be moved from implanted section 14 to external section 12 and vice versa.

In an exemplary embodiment, microphone 120 may be configured to pick up environmental sounds. In an exemplary embodiment, microphone 120 may include a microphone placed at an ear entrance of an implantee or a built-in microphone within sound processor 122, or any other suitable microphones for detecting sounds and speeches that are to be heard by an implantee. In an exemplary embodiment, sound processor 122 may be coupled with microphone 120 and may receive the picked up environmental sounds. In an exemplary embodiment, sound processor 122 may be configured to process a picked up environmental sound, in accordance with a sound processing program, to detect audible speech and to output the audible speech as processed audio signals. In an exemplary embodiment, sound processor 122 may be implemented by an earpiece that may be positioned behind an ear of an implantee, or alternatively may be implemented in the form of a wearable gadget.

In an exemplary embodiment, audible signals along with stimulation parameters and power signals from sound processor 122 may be transmitted to receiver/stimulator 140 by utilizing transmitter 124. In an exemplary embodiment, transmitter 124 may be configured to wirelessly send or transmit the stimulation parameters from external section 12 to implanted section 14. In an exemplary embodiment, transmitter 124 may be implemented in the form of a transmitter coil that may wirelessly transmit electrical signals to receiver/stimulator 140 via a radio frequency link.

In an exemplary embodiment, receiver/stimulator 140 may include an implantable receiving antenna that may receive stimulation parameters or power signals that may be transmitted by transmitter 124. In an exemplary embodiment, the implantable receiving antenna of receiver/stimulator 140 may be implemented in the form of a coil or other similar wireless communication components. In an exemplary embodiment, receiver/stimulator 140 may further include an implantable stimulator that may be configured to apply stimulation to stimulation sites located along an auditory pathway of an implantee, based at least in part on the received stimulation parameters from sound processor 122. In other words, an implantable stimulator of receiver/stimulator 140 may generate electrical stimulation that may be representative of a processed audio signal received from sound processor 122, in accordance with the stimulation parameters that may also be received from sound processor 122. In an exemplary embodiment, the generated electrical stimulation may be applied to the auditory nerve of an implantee via lead 142.

In an exemplary embodiment, electrode array 1420 of lead 142 may be connected via an extended cable to receiver/stimulator 140 and may be configured to receive the generated electrical stimulation from receiver/stimulator 140 and apply the received electrical stimulation to nerve cells within the cochlea of an implantee and, thereby, stimulating the auditory nerve. In an exemplary embodiment, electrode array 1420 may include a plurality of electrodes that may be disposed along lead 142.

Since electrode array 1420 may be inserted into the cochlea of an implantee, an inflammation in tissue may occur and surround electrode array 1420, as is the case for most implanted devices. Such inflammation may interfere with proper function of electrode array 1420. To address this inflammation problem, anti-inflammatory drugs, such as corticosteroids, may be administered into the cochlea of an implantee to reduce inflammation. Accordingly, system 10 may further include a system or mechanism to deliver an anti-inflammatory drug to the cochlea of an implantee, such as drug delivery system 128.

In an exemplary embodiment, drug delivery system 128 may be implemented by providing a cochlear implant with a channel at a center of electrode array 1420, through which an anti-inflammatory drug may be pumped from a drug container to an exemplary cochlea of an implantee. In an exemplary embodiment, drug delivery system 128 may be implemented by coating lead 142 with a silicone coating that may include an anti-inflammatory drug. The anti-inflammatory drug may be gradually released form the silicone coating within a period of one to three months.

Regardless of how drug delivery system 128 may be implemented, concentration of a delivered anti-inflammatory drug in the perilymph after implanting a cochlear implant must be measured as an important factor in determining the efficacy of the drug delivery. Accordingly, in an exemplary embodiment, lead 142 may further include electrochemical microsensor 1422 that may be coupled with electrochemical stimulator/analyzer 126. In an exemplary embodiment, electrochemical microsensor 1422 implanted within the cochlea of an implantee and electrochemical stimulator/analyzer 126 together may form an electrochemical system that may be configured to measure a concentration of an anti-inflammatory drug within the perilymph. In an exemplary embodiment, such association of an electrochemical system with a cochlear implant may allow for monitoring the concentration of anti-inflammatory drugs delivered to the inner ear of an implantee during and after the implantation of a cochlear implant.

In an exemplary embodiment, such electrochemical system formed by coupling implanted electrochemical microsensor 1422 and electrochemical stimulator/analyzer 126 may further be coupled to drug delivery system 128 to send feedback to drug delivery system 128. Such a feedback on drug concentration to drug delivery system 128 may allow for maintaining the concentration of an anti-inflammatory drug within the perilymph at a desired level, for example, by delivering more drug to the inner ear by drug delivery system 128 when the feedback from the electrochemical system indicates low levels of anti-inflammatory drug in the perilymph. In an exemplary embodiment, an effective amount of an anti-inflammatory drug in the perilymph may be between 20-40 μM and any level less than this range may be considered a low level of anti-inflammatory drug in the perilymph.

In an exemplary embodiment, electrochemical microsensor 1422 may be implemented comprising a three-electrode electrochemical sensor that may be connected to electrochemical stimulator/analyzer 126 via one or more electrically conductive connectors that may extend along lead 142 and may be connected to electrochemical stimulator/analyzer 126 in external section 12 of cochlear implant system 10. In an exemplary embodiment, sound processor 122 may additionally be used to selectively couple electrochemical stimulator/analyzer 126 with electrochemical microsensor 1422. For example, sound processor 122 may include a connection port coupled with electrochemical microsensor 1422 and electrochemical stimulator/analyzer 126 may be connected to electrochemical microsensor 1422 by plugging connectors of electrochemical stimulator/analyzer 126 into the connection port of sound processor 122.

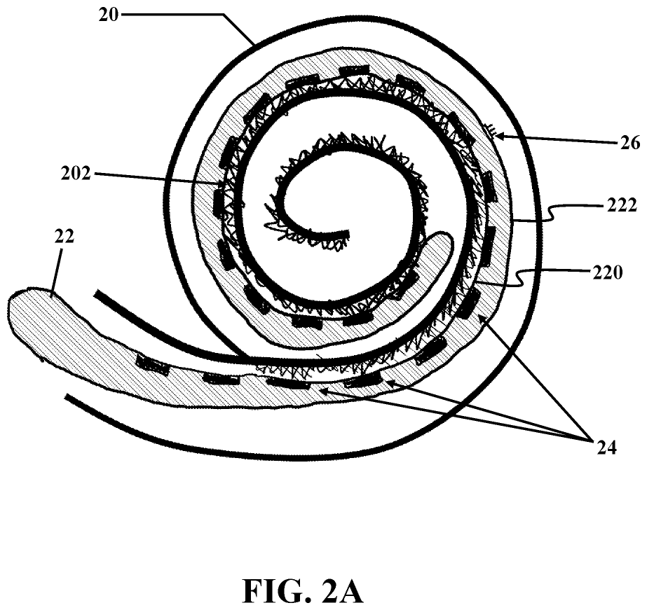

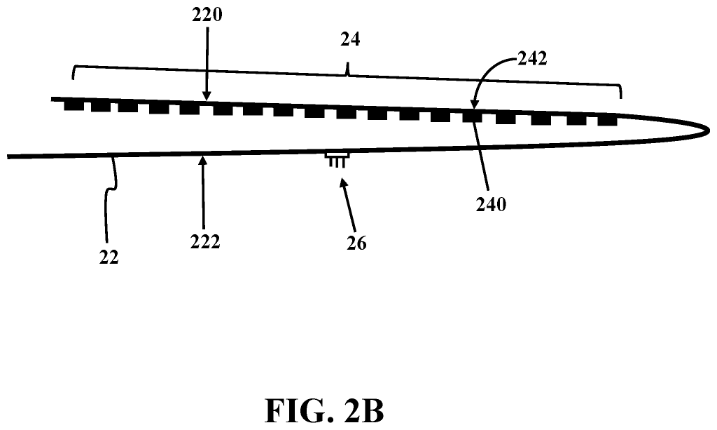

FIG. 2A illustrates a schematic top view of a human cochlea 20 and a lead 22 of a cochlear implant system inserted into human cochlea 20, consistent with one or more exemplary embodiments of the present disclosure. FIG. 2B illustrates a schematic structure of lead 22, consistent with one or more exemplary embodiments of the present disclosure. In an exemplary embodiment, lead 22 may be similar to lead 142 that may be implanted into human cochlea 20.

Human cochlea 20 may have a spiral structure that starts at a base end and spirals up to an apex. Auditory nerve tissue 202 is tonotopically organized within human cochlea 20. As used herein, tonotopic arrangement of auditory nerve tissue 202 may refer to the fact that low frequencies are encoded near the apex of human cochlea 20, while relatively high frequencies are encoded near the base end of human cochlea 20. In other words, different locations along human cochlea 20 may correspond to different respective perceived frequencies. Accordingly, lead 22 may include a plurality of electrodes 24 disposed along lead 22 that may be utilized for independently stimulating different locations within human cochlea 20 to create a hearing sensation.

In an exemplary embodiment, plurality of electrodes 24 may form an electrode array similar to electrode array 1420. Each electrode of plurality of electrodes 24 may separately be associated with an independent current source. This way, different stimulation current levels may be concurrently applied by different electrodes of plurality of electrodes 24 to multiple stimulation sites along human cochlea 20. In an exemplary embodiment, lead 22 may be made of a resiliently flexible material, such as silicone, such that when lead 22 may be inserted into human cochlea 20, it may flexibly spiral up into the spiral structure of human cochlea 20. In an exemplary embodiment, lead 22 may include an inner surface 220 and an opposing outer surface 222. As used here in, inner and outer are defined relative to the position of auditory nerve tissue 202 within human cochlea 20. Specifically, a surface of spiraled lead 22 positioned close to and facing toward auditory nerve tissue 202 is defined as inner surface 220 and the surface of lead 22 on an opposite side of lead 22 may be defined as opposing outer surface 222, which is facing away from auditory nerve tissue 202. In an exemplary embodiment, inner surface 220 may be adapted to be positioned at a surface of the modiolus of human cochlea 20 following the insertion of lead 22 into human cochlea 20. In other words, inner surface 220 may be adapted to be positioned at a surface of auditory nerve tissue 202 within human cochlea 20, following the insertion of lead 22 into human cochlea 20. In an exemplary embodiment, plurality of electrodes 24 may be supported within lead 22 and may be disposed along lead 22, such that a contact surface of each electrode of plurality of electrodes 24 may be aligned with inner surface 220 of lead 22. For example, a contact surface 242 of an electrode 240 may be aligned with inner surface 220 of lead 20.

In an exemplary embodiment, all connectors and cables that may be utilized for connecting plurality of electrodes 24 to a receiver/stimulator of a cochlear implant, such as receiver/stimulator 140 may run through the internal structure of lead 22. For example, lead 22 may further include an inner channel that may be used for running the cables and conductive connectors. Such cables and conductive connectors are not illustrated and labeled for simplicity.

In an exemplary embodiment, lead 22 may further include an electrochemical microsensor 26 that may be attached to outer surface 222 of lead 22. In an exemplary embodiment, electrochemical microsensor 26 may be structurally and functionally similar to electrochemical microsensor 1422. In an exemplary embodiment, electrochemical microsensor 26 may be connected to an electrochemical stimulator/analyzer, such as electrochemical stimulator/analyzer 126. In an exemplary embodiment, electrochemical microsensor 26 together with the electrochemical stimulator/analyzer may form an electrochemical system that may be configured for detecting a concentration of an anti-inflammatory drug delivered into human cochlea 20. In an exemplary embodiment, electrochemical microsensor 26 may be attached to an opposing surface of lead 22 relative to the surface on which plurality of electrodes 24 may be disposed, in order to avoid any interference between electrochemical microsensor 26 and plurality of electrodes 24. In other words, electrochemical microsensor 26 may be attached to outer surface 222 opposite inner surface 220, on which plurality of electrodes 24 may be arranged.

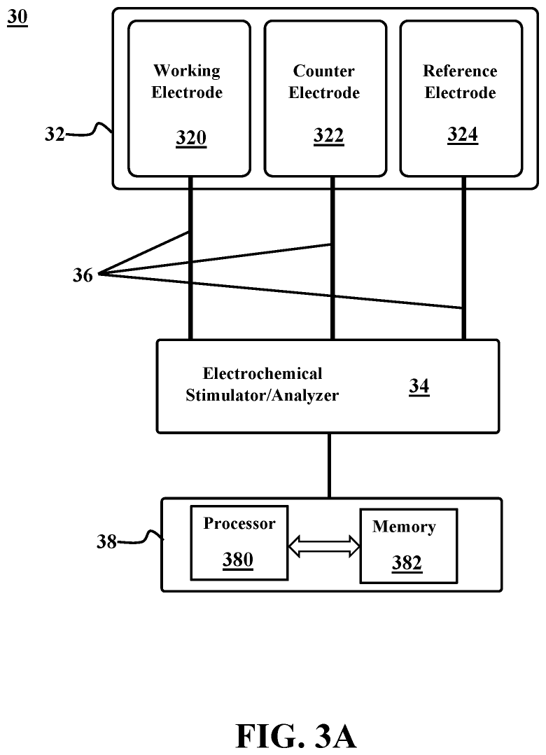

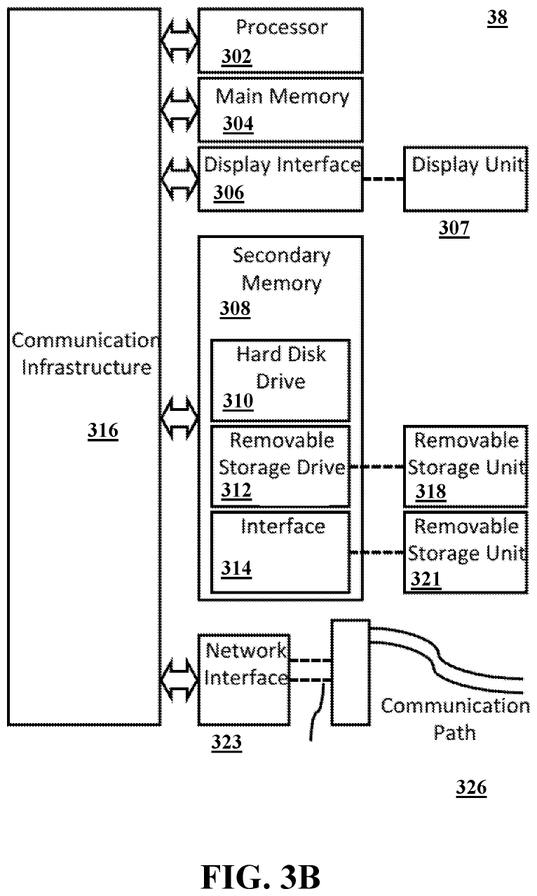

FIG. 3A illustrates a box diagram of an electrochemical system 30, consistent with one or more exemplary embodiments of the present disclosure. In an exemplary embodiment, an exemplary electrochemical microsensor, such as electrochemical microsensors 1422 and 26, together with electrochemical stimulator/analyzer 126 may form an electrochemical system similar to electrochemical system 30.

In an exemplary embodiment, electrochemical system 30 may include a sensor 32, an electrochemical stimulator/analyzer 34, and an array of electrically conductive connectors 36. In an exemplary embodiment, sensor 32 may be similar to electrochemical microsensors 1422 or 26. In an exemplary embodiment, sensor 32 may be put in contact with the perilymph inside a human cochlea, such as human cochlea 20. Perilymph may refer to an extracellular fluid located within the inner ear or cochlea. As used herein, putting sensor 32 in contact with the perilymph may refer to sensor 32 being placed in and exposed to the extracellular fluid within a human cochlea, which is referred to herein as cochlear fluid. In an exemplary embodiment, sensor 32 may include an integrated three-electrode array that may include a working electrode 320, a counter electrode 322, and a reference electrode 324. In an exemplary embodiment, electrochemical stimulator/analyzer 34 may be connected to sensor 32 via array of electrically conductive connectors 36. In an exemplary embodiment, electrochemical stimulator/analyzer 34 may be configured to measure electrochemical responses from working electrode 320.

In an exemplary embodiment, electrochemical stimulator/analyzer 34 may be a device, such as a potentiostat capable of measuring differential pulse voltammetry (DPV) diagrams. In an exemplary embodiment, electrochemical system 30 may further include a processing unit 38 that may be utilized to record and analyze electrochemical measurements made by electrochemical stimulator/analyzer 34. Processing unit 38 may further be utilized to control electrochemical stimulations that may be carried out by electrochemical stimulator/analyzer 34.

In an exemplary embodiment, processing unit 38 may be implemented as a programmable logic controller with at least one processor 380, and at least one memory 382 that may be coupled to at least one processor 380. In an exemplary embodiment, at least one memory 382 may store executable instructions to urge at least one processor 380 to perform operations including receiving a measured electrochemical response from electrochemical stimulator/analyzer 34, determining a peak current for the measured electrochemical response, receiving a calibration relationship between the peak current and concentration of the corticosteroid in the cochlear fluid, determining the concentration of the corticosteroid based at least in part on the determined peak current of the measured electrochemical response utilizing the received calibration relationship between the peak current and concentration of the corticosteroid in the cochlear fluid. FIG. 3B illustrates a high-level functional block diagram of processing unit 38, consistent with one or more exemplary embodiments of the present disclosure. For example, executable instructions for determining the concentration of the corticosteroid based at least in part on the determined peak current of the measured electrochemical response utilizing the received calibration relationship between the peak current and concentration of the corticosteroid in the cochlear fluid may be implemented in processing unit 38 using hardware, software, firmware, tangible computer readable media having instructions stored thereon, or a combination thereof and may be implemented in one or more processing units or other processing systems.

If programmable logic is used, such logic may execute on a commercially available processing platform or a special purpose device. One ordinary skill in the art may appreciate that an embodiment of the disclosed subject matter can be practiced with various processing unit configurations, including multi-core multiprocessor systems, minicomputers, mainframe computers, computers linked or clustered with distributed functions, as well as pervasive or miniature computers that may be embedded into virtually any device.

For instance, a processing unit having at least one processor device and a memory may be used to implement the above-described embodiments. A processor device may be a single processor, a plurality of processors, or combinations thereof. Processor devices may have one or more processor “cores.”

An embodiment of the invention is described in terms of this example processing unit 38. After reading this description, it will become apparent to a person skilled in the relevant art how to implement the invention using other processing units and/or computer architectures. Although operations may be described as a sequential process, some of the operations may in fact be performed in parallel, concurrently, and/or in a distributed environment, and with program code stored locally or remotely for access by single or multi-processor machines. In addition, in some embodiments the order of operations may be rearranged without departing from the spirit of the disclosed subject matter.

Processor device 304 may be a special purpose or a general-purpose processor device. As will be appreciated by persons skilled in the relevant art, processor device 304 may also be a single processor in a multi-core/multiprocessor system, such system operating alone, or in a cluster of processing units operating in a cluster or server farm. Processor device 304 may be connected to a communication infrastructure 316, for example, a bus, message queue, network, or multi-core message-passing scheme.

In an exemplary embodiment, processing unit 38 may include a display interface 306, for example a video connector, to transfer data to a display unit 307, for example, a monitor. Processing unit 38 may also include a main memory 304, for example, random access memory (RAM), and may also include a secondary memory 308. Secondary memory 308 may include, for example, a hard disk drive 310, and a removable storage drive 312. Removable storage drive 312 may include a floppy disk drive, a magnetic tape drive, an optical disk drive, a flash memory, or the like. Removable storage drive 312 may read from and/or write to a removable storage unit 318 in a well-known manner. Removable storage unit 318 may include a floppy disk, a magnetic tape, an optical disk, etc., which may be read by and written to by removable storage drive 312. As will be appreciated by persons skilled in the relevant art, removable storage unit 318 may include a computer usable storage medium having stored therein computer software and/or data.

In alternative implementations, secondary memory 308 may include other similar means for allowing computer programs or other instructions to be loaded into processing unit 38. Such means may include, for example, a removable storage unit 321 and an interface 314. Examples of such means may include a program cartridge and cartridge interface (such as that found in video game devices), a removable memory chip (such as an EPROM, or PROM) and associated socket, and other removable storage units 321 and interfaces 314 which allow software and data to be transferred from removable storage unit 321 to processing unit 38.

Processing unit 38 may also include a communications interface 323. Communications interface 323 allows software and data to be transferred between processing unit 38 and external devices. Communications interface 323 may include a modem, a network interface (such as an Ethernet card), a communications port, a PCMCIA slot and card, or the like. Software and data transferred via communications interface 323 may be in the form of signals, which may be electronic, electromagnetic, optical, or other signals capable of being received by communications interface 323. These signals may be provided to communications interface 323 via a communications path 326. Communications path 326 carries signals and may be implemented using wire or cable, fiber optics, a phone line, a cellular phone link, an RF link or other communications channels.

In this document, the terms “computer program medium” and “computer usable medium” are used to generally refer to media such as removable storage unit 318, removable storage unit 321, and a hard disk installed in hard disk drive 310. Computer program medium and computer usable medium may also refer to memories, such as main memory 304 and secondary memory 308, which may be memory semiconductors (e.g. DRAMs, etc.).

Computer programs (also called computer control logic) are stored in main memory 304 and/or secondary memory 308. Computer programs may also be received via communications interface 323. Such computer programs, when executed, enable processing unit 38 to implement different embodiments of the present disclosure as discussed herein. In particular, the computer programs, when executed, enable processor device 304 to implement the processes of the present disclosure. Accordingly, such computer programs represent controllers of processing unit 38. Where an exemplary embodiment of a method for measuring concentration of corticosteroid may be implemented using software, the software may be stored in a computer program product and loaded into processing unit 38 using removable storage drive 414, interface 420, and hard disk drive 412, or communications interface 424.

Embodiments of the present disclosure also may be directed to computer program products including software stored on any computer useable medium. Such software, when executed in one or more data processing device, causes a data processing device to operate as described herein. An embodiment of the present disclosure may employ any computer useable or readable medium. Examples of computer useable mediums include, but are not limited to, primary storage devices (e.g., any type of random-access memory), secondary storage devices (e.g., hard drives, floppy disks, CD ROMS, ZIP disks, tapes, magnetic storage devices, and optical storage devices, MEMS, nanotechnological storage device, etc.).

The embodiments have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

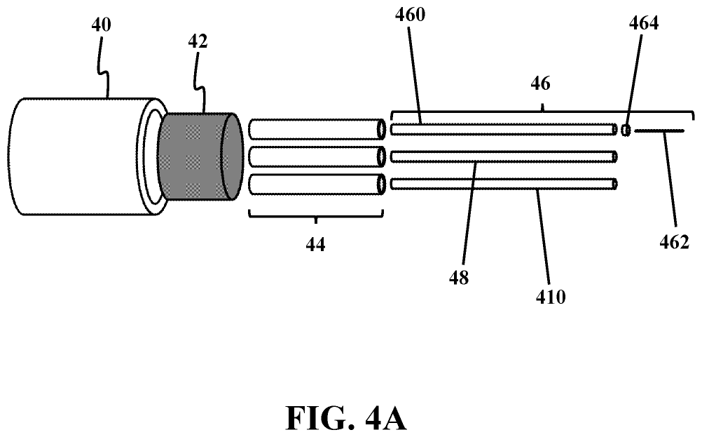

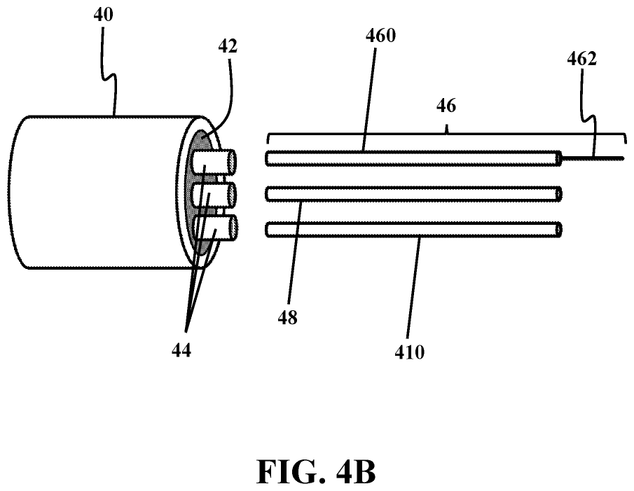

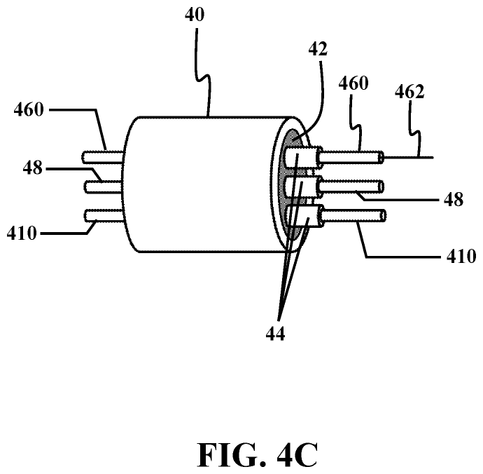

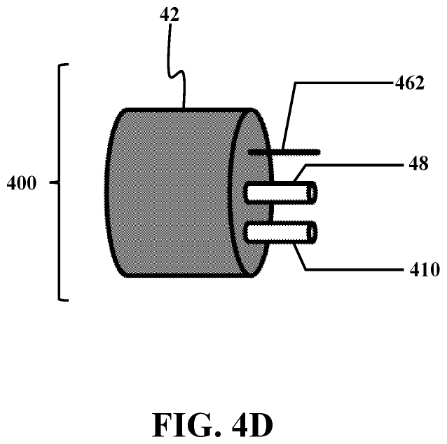

FIG. 4D illustrates a schematic perspective view of an electrochemical microsensor 400, consistent with one or more exemplary embodiments of the present disclosure. In an exemplary embodiment, sensor 32 may be similar to electrochemical microsensor 400.

In an exemplary embodiment, electrochemical microsensor 400 may include a working electrode 462, a counter electrode 48, a reference electrode 410 that may be disposed within and supported by a flexible holder 42. In an exemplary embodiment, working electrode 320 may be similar to working electrode 462, counter electrode 322 may be similar to counter electrode 48, and reference electrode 324 may be similar to reference electrode 410.

In an exemplary embodiment, working electrode 462 may include a carbon microfiber with a diameter of 5 micron to 10 micron that may extend out of flexible holder 42 by a length of 50 microns to 5000 microns. In an exemplary embodiment, counter electrode 48 may include a platinum wire with a diameter of 10 microns to 200 microns that may extend out of flexible holder 42 by a length of 50 microns to 5000 microns. In an exemplary embodiment, reference electrode 410 may include an Ag/AgCl wire with a diameter of 10 microns to 200 microns that may extend out of flexible holder 42 by a length of 50 microns to 5000 microns.

In an exemplary embodiment, fabricating working electrode 462, counter electrode 48, and reference electrode 410 with sizes within the ranges described in the preceding paragraph may allow for eliminating the negative effects, such as electrical interferences, due to the presence of electrochemical microsensor 400 within the cochlea of an implantee. Applying potentials to electrochemical microsensor 400 while performing electrochemical measurements may interfere with the performance of plurality of electrodes 24. Since the cochlear fluid is electrically conductive, the presence of electrochemical microsensor 400 within human cochlea may affect the distribution of the electric field on plurality of electrodes 24 and it may consequently alter the current density distribution on the auditory neurons.

In an exemplary embodiment, flexible holder 42 may include a flexible septum that may be made of a resiliently flexible material, such as pure silicone, pure silicone laminated to polytetrafluoroethylene (PTFE), and pure silicone sandwiched between two layers of PTFE. In an exemplary embodiment, a first portion of each of working electrode 462, counter electrode 48, and reference electrode 410 may penetrate into and be positioned within flexible holder 42 and a second remaining portion of each of working electrode 462, counter electrode 48, and reference electrode 410 may extend out of flexible holder 42, as discussed in the preceding paragraph.

FIGS. 4A-4D illustrate an exemplary electrochemical sensor at different stages of manufacturing process, consistent with one or more exemplary embodiments of the present disclosure. In an exemplary embodiment, a manufacturing process of electrochemical microsensor 400 may be described using the embodiments shown in FIGS. 4A-4D.

In an exemplary embodiment, working electrode 462 may be a part of a working electrode assembly 46 that may further include a steel wire 460. In an exemplary embodiment, working electrode 462 may be attached to one end of steel wire 460 by utilizing an electrical conductive glue, such as a silver paste 464. In an exemplary embodiment, steel wire 460 may include a steel wire with a diameter between 10 micron and 200 micron.

In an exemplary embodiment, to form electrochemical microsensor 400, working electrode assembly 46, counter electrode 48, and reference electrode 410 may be inserted into flexible holder 42. In an exemplary embodiment, flexible holder 42 may be made of a flexible material, such as silicone that may be capable of providing a leak-free seal and handling repeated puncturing. In an exemplary embodiment, flexible holder 42 may be made of at least one of pure silicone, pure silicone laminated to polytetrafluoroethylene (PTFE), and pure silicone sandwiched between two layers of PTFE. Such materials allow for flexible holder 42 to quickly restore its initial form even after being punctured.

In an exemplary embodiment, one approach for inserting working electrode assembly 46, counter electrode 48, and reference electrode 410 into flexible holder 42 may include placing flexible holder 42 within a support structure 40 that may hold flexible holder 42 in place, as for example, illustrated in FIG. 4B. In other words, flexible holder 42 may be tightly fit into support structure 40 such that flexible holder 42 may not have any unwanted translational or rotational movements relative to support structure 40. After that, three cannulas 44, each corresponding to one of working electrode assembly 46, counter electrode 48, and reference electrode 410 may be pushed into flexible holder 42 as, for example, illustrated in FIG. 4B. In an exemplary embodiment, three cannulas 44 may create three pathways within flexible holder 42 for working electrode assembly 46, counter electrode 48, and reference electrode 410 to pass through. In a next step, working electrode assembly 46, counter electrode 48, and reference electrode 410 may be inserted into three cannulas 44, as illustrated in FIG. 4C. Finally, three cannulas 44 may be pulled out of flexible holder 42 and flexible holder 42 may return to its initial form and thereby tightly surrounds the outer surfaces of working electrode assembly 46, counter electrode 48, and reference electrode 410. After that, flexible holder 42 may be removed from support structure 40 and electrochemical microsensor 400 may be formed.

In an exemplary embodiment, working electrode 462 may include a carbon microfiber with a diameter of 5 micron to 10 micron, which may be attached to steel wire 460 by utilizing silver paste 464. In an exemplary embodiment, working electrode 462 may be fabricated by first washing a carbon fiber bundle with acetone and deionized water and then separating a single carbon fiber from the cleaned bundle. An exemplary single carbon fiber separated this way may then be cut into a desired length and may be used as working electrode 462.

In an exemplary embodiment, an exemplary carbon fiber that may be utilized as working electrode 462 may be subjected to a surface treatment that may help amplify the signals generated by electrochemical microsensor 400. For example, an exemplary carbon fiber may be placed in a 0.5 M sulfuric acid solution and a potential of −2 V may be applied to the exemplary carbon fiber for 300 seconds. This way, an electrolysis of the solvent may occur on the surface of the exemplary carbon fiber, where gases released as the products of such electrolysis process may increase nanometric grooves and porosity of the exemplary carbon fiber.

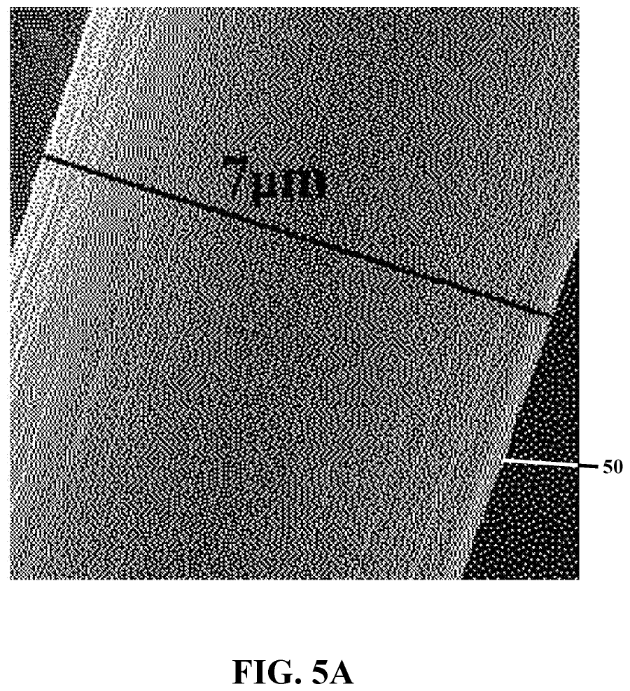

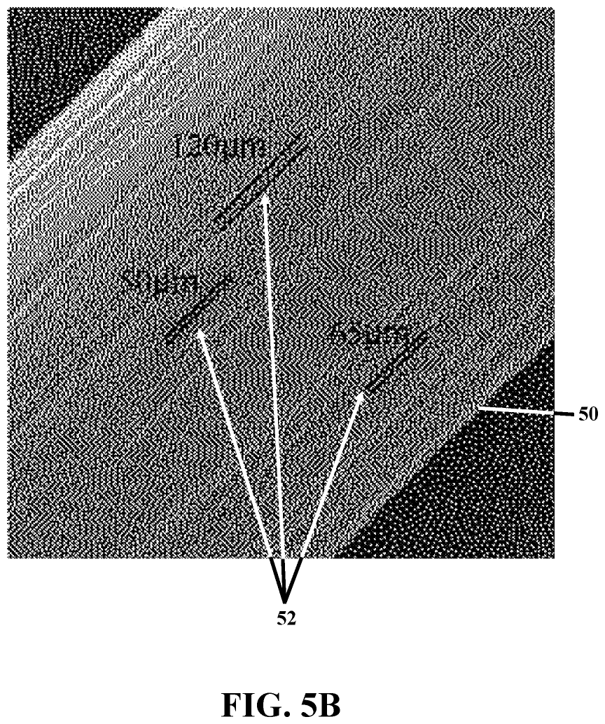

FIG. 5A illustrates a scanning electron microscope (SEM) image of a single carbon fiber 50 before surface treatment, consistent with one or more exemplary embodiments of the present disclosure. FIG. 5B illustrates an SEM of single carbon fiber 50 after surface treatment, consistent with one or more exemplary embodiments of the present disclosure. In an exemplary embodiment. Carbon fiber 50 has a diameter of 7 microns as evident in FIGS. 5A and 5B. In an exemplary embodiment, carbon fiber 50 may be placed in a 0.5 M sulfuric acid solution and a potential of −2 V may be applied to the exemplary carbon fiber for 300 seconds. After such treatment, as evident from FIG. 5B, the number of grooves, such as grooves 52 may considerably increase.

In an exemplary embodiment, reference electrode 410 may include an Ag/AgCl reference electrode with a diameter of 10 micron to 200 micron. In an exemplary embodiment, reference electrode 410 may be fabricated by placing a silver wire into a 0.5 M hydrochloric acid solution and polarizing it by applying a potential difference of about 0.6 V for 30 minutes.

In an exemplary embodiment, the electrochemical parameters of an electrochemical system, such as electrochemical system 30 that may be utilized in association with a cochlear implant, as for example implemented by cochlear implant system 10, must be optimized for measuring the concentration of each specific anti-inflammatory drug. In an exemplary embodiment, the electrochemical responses from an electrochemical microsensor, such as sensor 32 that may be implemented by electrochemical microsensor 400 may include DPV diagrams obtained by utilizing an electrochemical analyzer, such as electrochemical stimulator/analyzer 34. In an exemplary embodiment, many factors, such as electrode structure and material, applied potential, time intervals, pulse amplitudes, pulse time, and potential steps may be considered as the most influential parameters in the electrochemical measurements of electrochemical system 30.

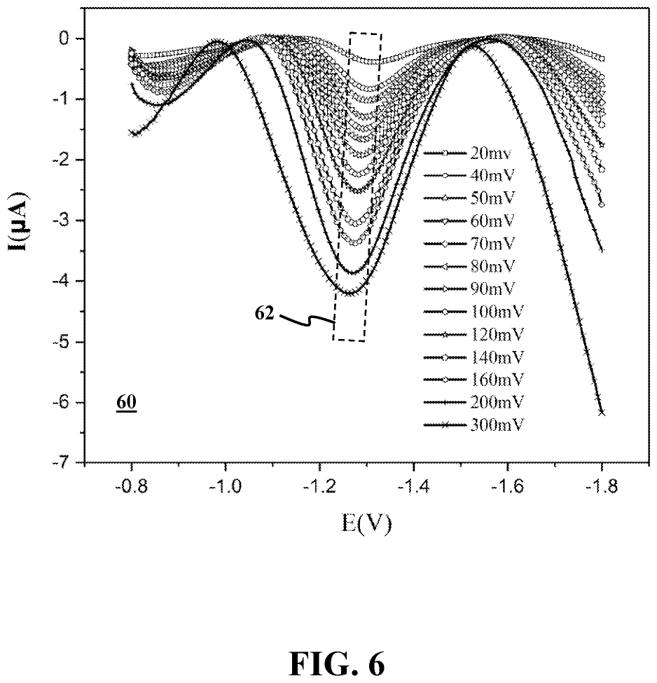

FIG. 6 illustrates differential pulse voltammetry (DPV) voltammograms 60 obtained by electrochemical system 30 for measuring 50 μM of dexamethasone in an artificial perilymph solution at different pulse amplitudes, consistent with one or more exemplary embodiments of the present disclosure. DPV voltammograms 60 may be obtained by placing electrochemical microsensor 400 within an artificial perilymph solution that contains 50 μM of dexamethasone and applying a potential to working electrode 462 relative to reference electrode 410 in a range of −0.8 to −1.8 V. As evident in FIG. 6, in an exemplary embodiment, the changes in peak currents 62 show that the peak current may increase with an increase in the pulse amplitude in a range of 20-300 mV with the Ip value of 0.3-4.2 μA for 50 μM of dexamethasone. The variation in the peak of potential may be negligible but the peak width increases with increasing the pulse amplitude since a large peak width is not suitable for the analysis of lower concentrations of dexamethasone. Accordingly, in an exemplary embodiment, the electrochemical responses from electrochemical microsensor 400 may include DPV diagrams measured at a pulse amplitude between 10 mV and 500 mV.

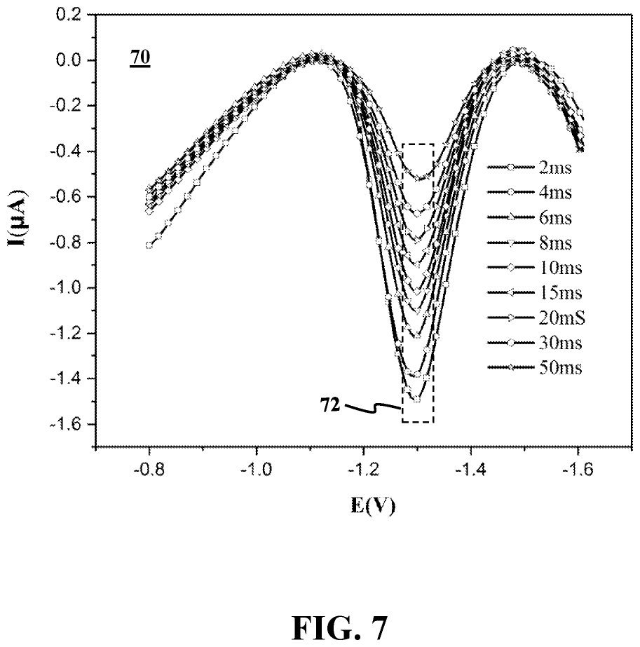

FIG. 7 illustrates differential pulse voltammetry (DPV) voltammograms 70 obtained by electrochemical system 30 for measuring 50 μM of dexamethasone in an artificial perilymph solution at different pulse times, consistent with one or more exemplary embodiments of the present disclosure. As evident in FIG. 7, in an exemplary embodiment, the changes in peak currents 72 show that the peak current may decrease with an increase in the pulse time in a range of 2-50 ms without any change in the potential peak or peak width. The highest peak current is observed for a pulse time of approximately 2 ms. Accordingly, in an exemplary embodiment, the electrochemical responses from electrochemical microsensor 400 may include DPV diagrams measured at a pulse time between 1 ms and 100 ms.

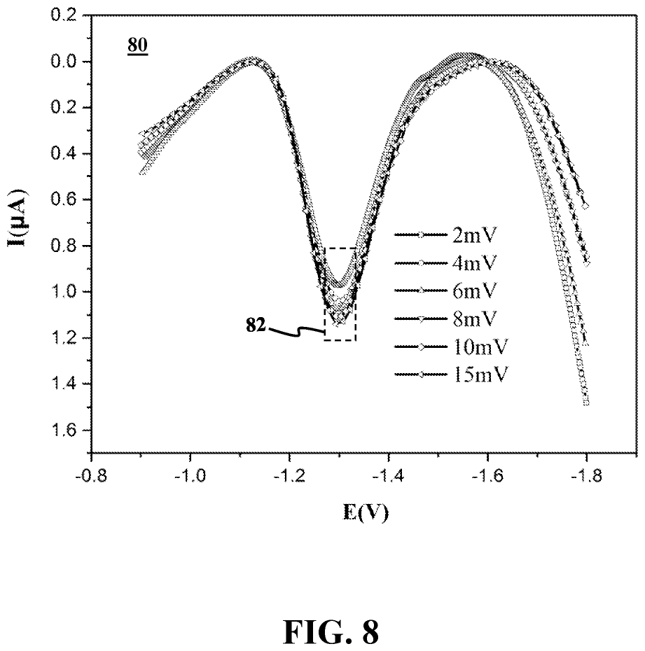

FIG. 8 illustrates differential pulse voltammetry (DPV) voltammograms 80 obtained by electrochemical system 30 for measuring 50 μM of dexamethasone in an artificial perilymph solution at different potential steps, consistent with one or more exemplary embodiments of the present disclosure. As evident in FIG. 8, in an exemplary embodiment, the changes in peak currents 82 show that the peak current may increase with an increase in the potential step. The highest peak current is observed for a potential step of approximately 15 mV. Accordingly, in an exemplary embodiment, the electrochemical responses from electrochemical microsensor 400 may include DPV diagrams measured at a potential step between 1 mV and 30 mV.

In an exemplary embodiment, based on the studies carried out as described in the preceding paragraphs, an exemplary electrochemical stimulator/analyzer of an exemplary electrochemical system that may be utilized within an exemplary cochlear implant system, such as electrochemical stimulator/analyzer 34, may be configured to measure electrochemical responses from an exemplary microsensor, such as sensor 32. In an exemplary embodiment, the aforementioned electrochemical responses from sensor 32 may include DPV diagrams that may be measured at a step potential between 1 mV and 300 mV, a pulse time between 1 ms and 100 ms, and a pulse amplitude between 10 mV and 500 mV.

### Example: Measuring Dexamethasone Concentration

In this example, an electrochemical system similar to electrochemical system 30, in which sensor 32 may be similar to electrochemical sensor 400 was evaluated for quantitative analysis of dexamethasone. In this example, electrochemical responses from electrochemical sensor 400 was measured by electrochemical stimulator/analyzer 34 at a step potential of 15 mV, a pulse time of 2 ms, and a pulse amplitude of 140 mV. The measurements were carried out in an artificial perilymph solution containing different amounts of dexamethasone.

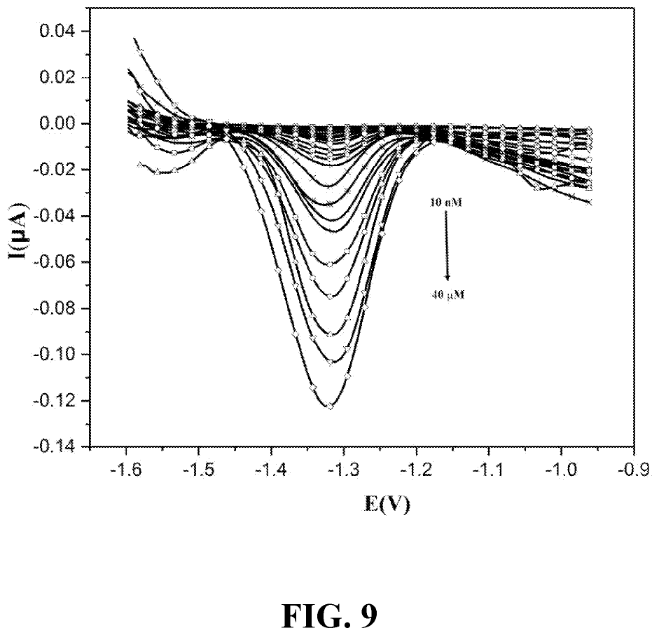

FIG. 9 illustrates DPV voltammograms obtained from electrochemical sensor 400 for various artificial perilymph solutions containing different amounts of dexamethasone, consistent with one or more exemplary embodiments of the present disclosure. In an exemplary embodiment, DPV voltammograms were obtained for concentrations of dexamethasone ranging between 10 nM and 40 μM. This range corresponds to the usual concentration of administered dexamethasone in a human cochlea. The sensitivity of the DPV method performed by electrochemical system 30 was found to be 16 (μAμM⁻¹ cm⁻²) and the limit of detection for electrochemical system 30 was approximately 4×10⁻⁹ M.

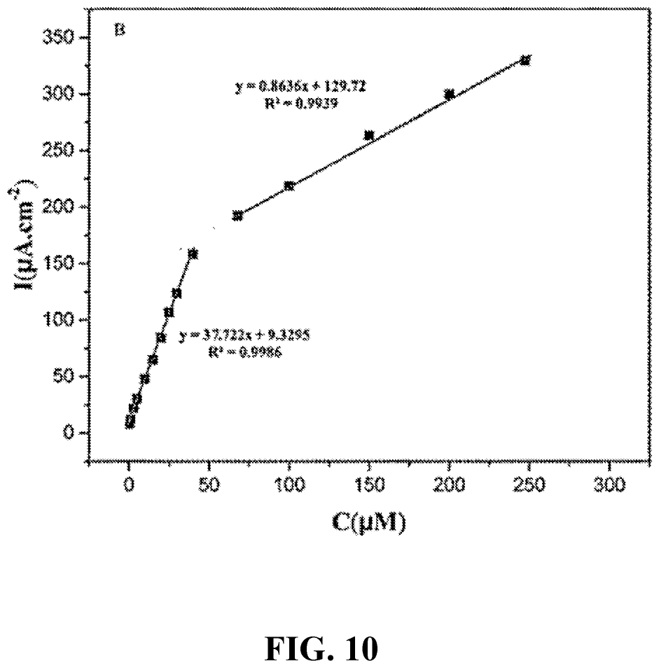

FIG. 10 illustrates calibration plots of peak currents versus various concentrations of dexamethasone in artificial perilymph solutions, consistent with one or more exemplary embodiments of the present disclosure. In this example, peak current of each DPV voltammogram was plotted versus a corresponding concentration of dexamethasone for which that DPV voltammogram was obtained. This way a calibration relationship may be obtained between the peak current and the concentration of dexamethasone within the perilymph. In an exemplary embodiment, an electrochemical system similar to electrochemical system 30 may be configured to measure dexamethasone concentration in a cochlear fluid based at least in part on a current peak measured by electrochemical stimulator/analyzer 34 utilizing an established calibration relationship between the peak current and the concentration of dexamethasone.

The embodiments have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for description and not of limitation, such that the terminology or phraseology of the present specification is to be interpreted by the skilled artisan in light of the teachings and guidance.

The breadth and scope of the present disclosure should not be limited by any of the above-described exemplary embodiments but should be defined only in accordance with the following claims and their equivalents.

Throughout this specification and the claims which follow, unless the context requires otherwise, the word “comprise”, and variations such as “comprises” or “comprising”, will be understood to imply the inclusion of a stated integer or step or group of integers or steps but not to the exclusion of any other integer or step or group of integers or steps.

Moreover, the word “substantially” when used with an adjective or adverb is intended to enhance the scope of the particular characteristic; e.g., substantially planar is intended to mean planar, nearly planar and/or exhibiting characteristics associated with a planar element. Further use of relative terms such as “vertical”, “horizontal”, “up”, “down”, and “side-to-side” are used in a relative sense to the normal orientation of the apparatus.

What is claimed is:

1. An electrochemical system for a cochlear implant, the electrochemical system comprising:
a microsensor attached to an electrode array of the cochlear implant, the microsensor configured to be put in contact with a cochlear fluid, the microsensor comprising:
a working electrode comprising a carbon microfiber with a diameter of 5 micron to 10 micron;
a reference electrode comprising an Ag/AgCl wire with a diameter of 10 micron to 100 micron; and
a counter electrode comprising a platinum wire with a diameter of 10 micron to 100 micron;
an electrochemical stimulator/analyzer, the electrochemical stimulator/analyzer configured to measure electrochemical responses from the microsensor; and
an array of electrically conductive connectors, the microsensor connected to the electrochemical stimulator/analyzer via the array of electrically conductive connectors,
wherein the electrochemical system is configured to measure the corticosteroid concentration in the cochlear fluid.

2. An electrochemical system for a cochlear implant, the electrochemical system comprising:
a microsensor attached to an electrode array of the cochlear implant, the microsensor configured to be put in contact with a cochlear fluid, the microsensor comprising:
a working electrode;
a reference electrode; and
a counter electrode;
an electrochemical stimulator/analyzer, the electrochemical stimulator/analyzer configured to measure an electrochemical response from the microsensor; and
an array of electrically conductive connectors, the microsensor connected to the electrochemical stimulator/analyzer via the array of electrically conductive connectors,
wherein the electrochemical system is configured to measure the corticosteroid concentration in the cochlear fluid.

3. The electrochemical system of claim 2, further comprising a processing unit coupled with the electrochemical stimulator/analyzer, the processing unit comprising:
at least one processor; and
at least one memory coupled to the at least one processor, the at least one memory storing executable instructions to urge the at least one processor to:
receive the measured electrochemical response from the electrochemical stimulator/analyzer;
receive a calibration relationship between a peak current of the measured electrochemical response and the corticosteroid concentration in the cochlear fluid; and
calculate the corticosteroid concentration in the cochlear fluid based at least in part on the measured electrochemical response utilizing the received calibration relationship between a peak current of the measured electrochemical response and the corticosteroid concentration in the cochlear fluid.

4. The electrochemical system of claim 3, wherein:
the working electrode comprises a carbon microfiber with a diameter of 5 micron to 10 micron;
the reference electrode comprises an Ag/AgCl wire with a diameter of 10 micron to 100 micron, and
the counter electrode comprises a platinum wire with a diameter of 10 micron to 100 micron.

5. The electrochemical system of claim 4, wherein the microsensor further comprises a holding member holding the working electrode, the reference electrode, and the counter electrode, the holding member comprising a septum made of a resiliently flexible material, a first portion of each of the working electrode, the reference electrode, and the counter electrode penetrated into and positioned within the septum, a second portion of each of the working electrode, the reference electrode, and the counter electrode extended out of the septum.

6. The electrochemical system of claim 5, wherein the second portion of each of the working electrode, the reference electrode, and the counter electrode extends out of the septum by a length of 50 micron to 500 micron.

7. The electrochemical system of claim 5, wherein the working electrode further comprises a steel wire, a first end of the steel wire penetrated into and disposed within the septum and a second opposing end of the steel wire attached to the carbon microfiber.

8. The electrochemical system of claim 7, wherein the steel wire comprises a wire with a diameter of 10 micron to 200 micron.

9. The electrochemical system of claim 5, wherein the resiliently flexible material comprises at least one of pure silicone, pure silicone laminated to polytetrafluoroethylene (PTFE), and pure silicone sandwiched between two layers of PTFE.

10. The electrochemical system of claim 3, wherein the electrode array of the cochlear implant comprises:
an elongated lead, the elongated lead made of a resiliently flexible material configured to be inserted into cochlea of an implantee, an inner surface of the elongated lead adapted to be positioned at a surface of the modiolus of the cochlea following insertion of the electrode array; and
a plurality of electrodes supported within the elongated lead, a contact surface of each electrode of the plurality of electrodes aligned with the inner surface of the elongated lead,
wherein the microsensor is attached to an opposing outer surface of the elongated lead.

11. The electrochemical system of claim 10, wherein the microsensor further comprises a holding member holding the working electrode, the reference electrode, and the counter electrode, the holding member comprising a septum made of a resiliently flexible material, a first portion of each of the working electrode, the reference electrode, and the counter electrode penetrated into and positioned within the septum, a second portion of each of the working electrode, the reference electrode, and the counter electrode extended out of the septum.

12. The electrochemical system of claim 11, wherein:
the holding member comprises a cylindrical septum made of at least one of pure silicone, pure silicone laminated to polytetrafluoroethylene (PTFE), and pure silicone sandwiched between two layers of PTFE,
a first base end of the cylindrical septum attached to the opposing outer surface of the elongated lead, and
the second portion of each of the working electrode, the reference electrode, and the counter electrode extends out of an opposing second base end of the cylindrical septum.

13. The electrochemical system of claim 12, wherein the second portion of each of the working electrode, the reference electrode, and the counter electrode extends out of opposing second base end of the cylindrical septum by a length of 50 micron to 5000 micron.

14. The electrochemical system of claim 13, wherein:
the working electrode comprises a carbon microfiber with a diameter of 5 micron to 10 micron;
the reference electrode comprises an Ag/AgCl wire with a diameter of 10 micron to 200 micron, and
the counter electrode comprises a platinum wire with a diameter of 10 micron to 200 micron.

15. The electrochemical system of claim 10, wherein:
each electrode of the plurality of electrodes is connected to an implanted stimulator/receiver unit of the cochlear implant via at least two conductive wires, the at least two conductive wires extended along and disposed within the elongated lead, and
the array of electrically conductive connectors extended along and disposed within the elongated lead.

16. The electrochemical system of claim 3, wherein the electrochemical responses from the microsensor comprise differential pulse voltammetry diagrams measured at a step potential between 1 mV and 30 mV, a pulse time between 1 ms and 30 ms, and a pulse amplitude between 10 mV and 500 mV.
